(19) United States
(12) Patent Application Publication
(10) Pub. No.: US 2008/0146913 A1
(43) Pub. Date: Jun. 19, 2008

(54) THERMOCHROMIC DEVICES FOR VASCULAR ACCESS PROCEDURES

(75) Inventors: Arun Ranchod, Ellwood City, PA (US); Page Cedarholm, Pittsburgh, PA (US); Kevin P. Cowan, Allison Park, PA (US); Adrienne Fazio, Bethel Park, PA (US); David M. Griffiths, Pittsburgh, PA (US)

Correspondence Address: GREGORY L BRADLEY;MEDRAD INC, ONE MEDRAD DRIVE, INDIANOLA, PA, 15051, US

(73) Assignee: MEDRAD, INC., Indianola, PA (US)

(21) Appl. No.: 11/612,195

(22) Filed: Dec. 18, 2006

(51) Int. Cl. A61B 5/15

(52) U.S. Cl. 600/412; 600/417; 604/4.01

(57) ABSTRACT

A device for locating a venous structure of a patient includes a frame having a bottom surface and a top surface, an adhesive disposed on the bottom surface of the frame to allow the frame to be removably attached to skin or a patient, and one or more sections removably attached to the top surface of the frame. The sections are formed of a liquid crystal material that is sensitive to human skin temperature ranges.

### BACKGROUND OF THE INVENTION

This invention relates generally to vein visualization devices, and more specifically to thermochromic devices to locate or visualize venous structures or architecture in patients.

Intravenous (IV) access is problematic in many patients due to difficulty in finding and locating veins that are suitable. Many patients have veins that are not visible with the naked eye, or are beneath the surface of the skin so that they cannot be felt or seen. Patients with dark skin, and excess of subcutaneous fat, or with small or deep veins often fall into this category. In addition to patient discomfort, multiple attempts to obtain venous access increases the chance for infection.

As reported by InfraRed Imaging Systems, Inc. of Columbus, Ohio, vascular access procedures rank as the most commonly performed, invasive, medical procedure in the U.S., with over 1.4 billion procedures performed annually. These procedures also rank as the top patient complaint among clinical procedures. An overwhelming majority of vascular access procedures are performed without the aid of any visualization device and rely on what is observed through the patient's skin and by the clinician's ability to feel the vessel. Medical literature reports the following statistics: (1) a 28% first attempt IV failure rate in normal adults; (2) a 44% first attempt IV failure rate in pediatrics; (3) 43% of pediatric IVs require three or more insertion attempts; (4) a 23% to 28% incidence of extravasation/infiltration; (5) a 12% outright failure rate in cancer patients; and (6) 25% of hospital in-patients beyond three days experience difficult vascular access.

A number of prior art patents disclose thermochromic liquid crystal devices. For example, U.S. Pat. Nos. 4,140,016 and 5,130,828 describe thermochromic liquid crystal materials for temperature sensing devices.

In addition, U.S. Pat. No. 4,279,152 discloses a liquid crystal temperature indicator that provides a visual indication of whether the device is at a temperature above or below one or more threshold values.

Further, U.S. Pat. No. 4,015,591 describes a liquid crystal composition and a method of effecting venapuncture using the composition. U.S. Pat. No. 4,175,543 discloses a venapuncture method using a liquid crystal composition. U.S. Pat. No. 4,310,577 discloses a liquid crystal film laminate embodying an indicator component and a supportive and protective component removable therefrom. The contents of all of the above-listed U.S. patents are incorporated herein by reference.

Moreover, a number of products for locating veins are known or currently available. These include products utilizing liquid crystal thermal surface temperature measurement patches, such as the K-4000 Vena-Vue® Thermographic Vein Evaluator manufactured by Biosynergy, Inc. of Elk Grove Village, Ill. The K-4000 Vena-Vue® Thermographic Vein Evaluator is not configured to offer a convenient means for a clinician to both gain access to and visualize the patient's vasculature.

The inadequacies of current vascular access practices significantly compromise patient care and contribute to rising healthcare costs. Multiple access attempts and outright failures delay patient treatment, frustrate healthcare professionals, and increase the likelihood of downstream complications and expense.

### SUMMARY OF THE INVENTION

The invention provides an improved vein-visualization or vein-locating device that is intended for use during intravenous access medical procedures. In one aspect, the present invention provides a vein visualization device that is formed with one or more removable sections. The device allows a clinician to both visualize the patient's vasculature under the device and to gain access to the vasculature for insertion of, for example, a needle or catheter, into the vasculature.

The device could also be formed with one or more access holes to allow insertion of a needle or other access device into the patient's vasculature. In addition, the device could be formed of one or more strips or sections of liquid crystal material that can be removed from the device frame to permit venous access.

Preferably, the device is a patch having a base or frame with adhesive backing that adheres to the patient's skin. The patch includes one or more strips or sections of liquid crystal material that are removably connected to the base or frame.

In another aspect, the present invention provides a vein visualization device that is formed with one or more ends that are adapted to affix an access device, such as a needle or catheter, to the patient's skin after the access device is inserted into the patient's vasculature.

Preferably, the device is a patch-like that is attached to the patient's skin in proximity to the desired venous access site and the ends (which may be loose or dangling) are attached to the device. The ends may have adhesive on them for adhering to the access device and/or the patient's skin, such as gauze tape and the like.

In yet another aspect, the present invention provides a method for visualizing the vasculature of a patient by placing a temperature indication device on the patient's skin to indicate the patient's skin temperature, using the indicated temperature to choose an appropriate or suitable vein visualization device to use for a venous access procedure, and placing the vein visualization device on the patient's skin in proximity to the desired venous access site.

To induce blood flow and thereby warm the vasculature under the vein visualization device, the patient may squeeze her hand repeatedly, preferably using a hand-held grip or stress-relief ball.

In yet still another aspect, a device for locating a venous structure of a patient includes a frame having a bottom surface and a top surface, an adhesive disposed on the bottom surface of the frame to allow the frame to be removably attached to skin or a patient, and one or more sections removably attached to the top surface of the frame. The sections are formed of a liquid crystal material that is sensitive to human skin temperature ranges.

In another aspect, a device for locating a venous structure of a patient includes a frame having a bottom surface and a top surface, an adhesive disposed on the bottom surface of the frame to allow the frame to be removably attached to skin or a patient, a layer formed of a liquid crystal material attached to the top surface of the frame, and one or more end members connected to the frame for affixing a venous access device to the skin of the patient. The liquid crystal material is formulated to be sensitive to human skin temperature ranges.

In still another aspect, a method of locating a venous structure of a patient includes (1) placing a temperature indication device on the skin of the patient in proximity to a desired venous access site, (2) using the indicated skin temperature provided by the temperature indication device to select a vein visualization device that is sensitive to a temperature range that includes the indicated skin temperature, (3) placing the vein visualization device on the skin of the patient in proximity to the desired venous access site; and (4) inserting a venous access device into a desired portion of the venous structure that is indicated on the vein visualization device.

In still yet another aspect, a device for locating a venous structure of a patient includes a frame having a bottom surface and a top surface, an adhesive disposed on the bottom surface of the frame to allow the frame to be removably attached to skin of a patient, and a liquid crystal containing material that is sensitive to human skin temperature ranges. The liquid crystal containing material is formed as a single continuous sheet with numerous pores, or holes, in the continuum of the sheet. The pores allow direct access to the skin, while still providing an accurate visualization of patient vasculature location.

The present invention provides, in one preferred aspect, devices and methods that permit a clinician to both visualize the patient's vasculature under the device and to gain access to the vasculature for insertion of, for example, a needle or catheter, into the vasculature. The present invention aids clinicians in locating and accessing veins for various types of venous access procedures, such as for blood withdrawal, drug and fluid injections and antecubital IV placements.

### BRIEF DESCRIPTION OF THE DRAWINGS

The present invention, and its presently preferred and alternate embodiments, will be better understood by way of reference to the detailed description herebelow and to the accompanying drawings, wherein:

FIG. 1 shows isometric and cross-sectional views of a first embodiment of the present invention;

FIG. 2 shows the embodiment of FIG. 1 in use on a patient's arm;

FIG. 3 shows an isometric view of a second embodiment of the present invention;

FIG. 4a shows another isometric view of the second embodiment of the present invention;

FIG. 4b shows a detailed view of the device of FIG. 4a;

FIG. 5 shows an alternate embodiment of the device shown in FIGS. 3, 4a and 4b; and

FIG. 6 shows various views of a third embodiment of the present invention.

### DETAILED DESCRIPTION OF THE INVENTION

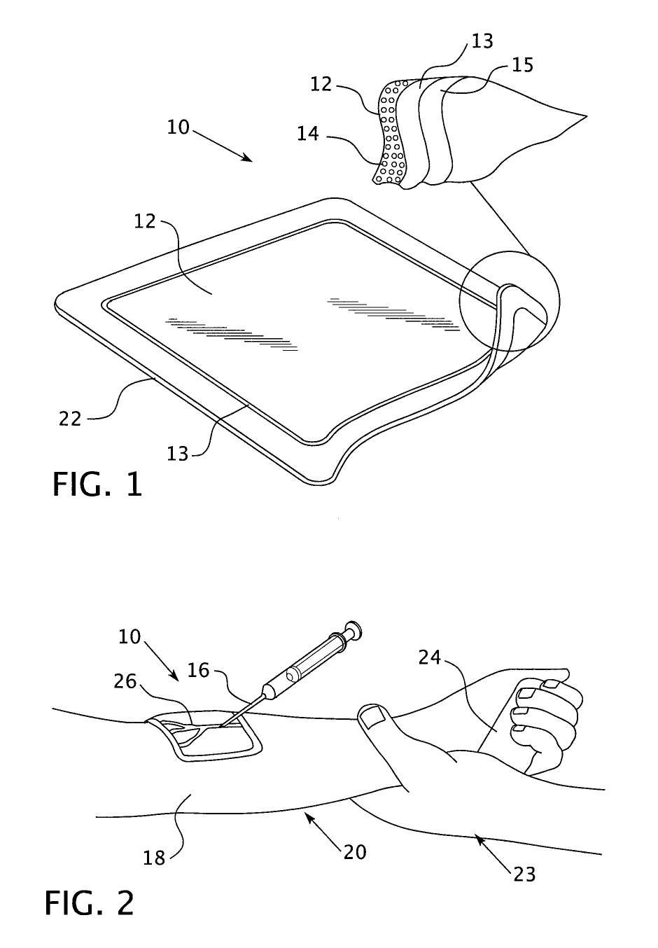

In one embodiment, as shown in FIGS. 1 and 2, the vein visualization device 10 of the present invention includes a thermochromic liquid crystal sheeting 12 (e.g., Mylar or other thin-film preparation) that is preferably receptive to temperatures within a range of 32-38 degrees Celsius with sensitivity approximately 0.2 degrees Celsius. The sheeting 12 may include numerous, uniformly-spaced small holes 14 through which a needle 16 may pass directly to the patient's skin 18. The device 10 also preferably includes a frame or base member 13 and an adhesive backing 15 (such as an adhesive fabric) that allows the edges 22 of the frame 13 to be removably attached to the patient's skin 18. Preferably, the bottom (i.e., skin-contacting side) of the device 10 may also be coated with a substance that is adapted to cool the skin 18 and promotes thermal transfer, such as an alcohol-based gel.

In a preferred embodiment, the present invention includes the device 10 and a hand-held grip 24, such as a “squishy ball”. The grip 24 may contain materials that, when squeezed, initiate a controlled exothermic reaction (i.e., it gets warm). Or, the patient could be requested to repetitively squeeze and release the grip 24 to induce blood flow through the patient's vasculature.

In an exemplary venous access procedure, as best represented in FIG. 2, the patient will present her arm 20 or other anatomical region including the target area for venous access. The clinician or medical personnel 23 will prep (e.g., with an alcohol wipe) the target area of her arm 20 and apply the device 10 to the target area. Once the device 10 is applied to the target area, the patient will squeeze the grip 24 to warm the patient's hand and induce greater blood flow near the surface of the hand. The thermochromic liquid crystal sheeting 12 will display areas of surface vasculature 26 because these areas will warm faster than the surrounding skin. Once the vasculature 26 is displayed on the sheeting 12, the clinician 23 will insert the needle 16, through the liquid crystal sheeting 12, into the indicated vasculature 26.

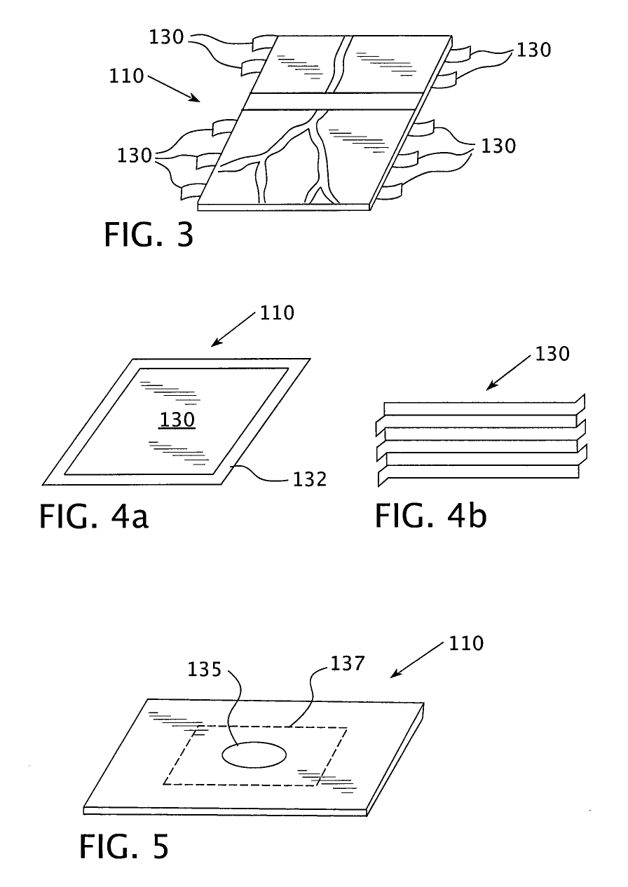

In another preferred embodiment, as best shown in FIGS. 3, 4a and 4b, the device 110 preferably includes one or more sections 130, preferably strips, of encapsulated enantiotropic cholesteric liquid crystalline phase material exhibiting a mesophase color change at temperature(s) corresponding to human skin temperatures. The device 110 allows clinicians to both visualize the target vasculature and gain access to that vasculature.

Having the device 110 configured in sections 130, as opposed to the solid sheet 12 described above, allows each individual section 130 to be removed from the patch frame 132 (see FIG. 3) individually. The patch frame 132 is preferably a circumference of gauze material, in any suitable shape or configuration, with an adhesive backing suitable for adhesion to human skin. This patch frame 132 structure allows the clinician to still visualize the remaining context of the vasculature in question, while providing an access point to that vasculature that does not pass through any portion of the patch (see FIG. 3). Thus, the clinician receives the benefits of substantial visualization while being afforded safe access to the surface of the skin.

In alternate embodiments, the removable sections 130 could be configured in various shapes, sizes and numbers. For example, the sections 130 could be formed in the shape of circles, squares, triangles and octagons. As shown in FIG. 5, the removable section 130 could be a single, circular section 135 located close to or in the center of the device 110. The circular section 135 could be formed in a perforated area 137 of the device 110. As best shown in FIGS. 3 and 4b, however, the sections 130 are preferably formed in a series of strips.

In another exemplary venous access procedure using the embodiment of the invention shown in FIGS. 3 and 4, the patient will present her arm or other anatomical region including the target area for venous access. The clinician or medical personnel will prep (e.g., with an alcohol wipe) the target area and apply the device 110 to the target area. Once the device 110 is applied to the target area, it will display areas of surface vasculature because these areas will be warmer than the surrounding skin. Once the vasculature is displayed on the device 110, the clinician will remove the section 130 from the frame 132 that overlies the venous structure to be accessed and will insert the needle through the opening in the device 110 and into the venous structure.

In yet another embodiment, the present invention is a vein visualization device 10, 110 used in conjunction with a currently-available skin temperature gauge (not shown), such as the Kwik-Skan® Liquid Crystal Temperature Monitor marketed by Trademark Medical, that may be used by the clinician to indicate the patient's current skin temperature. The indicated temperature may cause the clinician to either pre-treat the area of interest with heat or cold, or, alternatively, may cause the clinician to select a vein visualization device 10, 110 that is formulated more specifically for that particular temperature range.

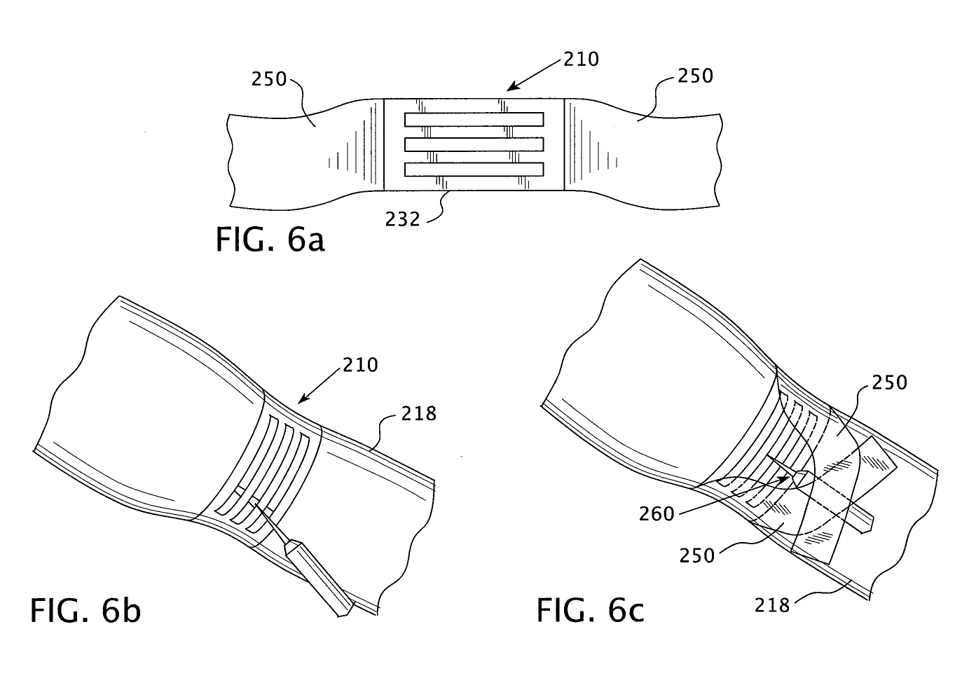

In another embodiment, as shown in FIG. 6, a vein visualization device 210 includes strips of adhesive 250 attached to the frame 232 that could be used to secure the IV access device 260 to the patient's skin 218, such as a needle or PICC line, after the vein has been located. This would add value to the clinician in that she would not have to separately tear off strips of adhesive tape to secure the IV access device after it has been successfully inserted into the venous structure of the patient.

In still another embodiment, the vein visualization device includes a frame having a bottom surface and a top surface, an adhesive disposed on the bottom surface of the frame to allow the frame to be removably attached to skin of a patient, and a liquid crystal containing material that is formed as a single continuous sheet with numerous pores, or holes, in the continuum of the sheet. The pores allow direct access to the skin, while still providing an accurate visualization of patient vasculature location.

The invention and embodiments described above can be used to aid various clinicians in locating and accessing veins for various types of injections and other medical procedures. One example is for chemotherapy infusion. Another example is blood donation. The most obvious example is for standard syringe injections or antecubital IV placements.

Although the present invention has been described in detail in connection with the above embodiments and/or examples, it is to be understood that such detail is solely for that purpose and that variations can be made by those skilled in the art without departing from the invention. The components and features of the various embodiments of the invention can be assorted or combined as appropriate for the application. The scope of the invention is indicated by the following claims rather than by the foregoing description. All changes and variations which come within the meaning and range of equivalency of the claims are to be embraced within their scope.

What is claimed is:

1. A device for locating a venous structure of a patient, comprising:
a frame having a bottom surface and a top surface;
an adhesive disposed on the bottom surface of the frame to allow the frame to be removably attached to skin or a patient; and
one or more sections removably attached to the top surface of the frame, the one or more sections being formed of a liquid crystal material that is sensitive to human skin temperature ranges.

2. The device of claim 1 wherein the frame comprises a gauze material.

3. The device of claim 1 wherein the frame is formed in the shape of a rectangle.

4. The device of claim 1 wherein the one or more sections are strips of liquid crystal materials.

5. The device of claim 1 wherein the frame defines a central open area that allows access to the skin of the patient.

6. The device of claim 1, further comprising one or more strips of adhesive connected to the frame for securing a venous access device to the skin of the patient.

7. The device of claim 1 wherein the one or more sections comprises a plurality of sections.

8. A device for locating a venous structure of a patient, comprising:
a frame having a bottom surface and a top surface;
an adhesive disposed on the bottom surface of the frame to allow the frame to be removably attached to skin or a patient;
a layer formed of a liquid crystal material attached to the top surface of the frame, the liquid crystal material being sensitive to human skin temperature ranges; and
one or more end members connected to the frame for affixing a venous access device to the skin of the patient.

9. The device of claim 8 wherein the one or more end members comprise an adhesive material for adhering the venous access device to the skin of the patient.

10. The device of claim 8 wherein the one or more end members each comprise a first end connected to the frame.

11. The device of claim 8 wherein the one or more end members comprise two end members.

12. The device of claim 8 wherein the layer is removably attached to the tope surface of the frame.

13. The device of claim 12 wherein the layer is formed of a plurality of sections.

14. A method of locating a venous structure of a patient, comprising:
placing a temperature indication device on the skin of the patient in proximity to a desired venous access site;
using the indicated skin temperature provided by the temperature indication device to select a vein visualization device that is sensitive to a temperature range that includes the indicated skin temperature;
placing the vein visualization device on the skin of the patient in proximity to the desired venous access site; and
inserting a venous access device into a desired portion of the venous structure that is indicated on the vein visualization device.

15. The method of claim 14, further comprising:
inducing blood flow to the venous structure.

16. The method of claim 15 wherein the step of inducing blood flow comprises having the patient squeeze a hand-held device.

17. The method of claim 14 wherein the venous access device is inserted through the vein visualization device into the venous structure.

18. The method of claim 14 wherein the vein visualization device comprises the device of any of claims 1-13.

19. The method of claim 14 wherein the vein visualization device comprises a frame and a layer of liquid crystal material connected to the frame.

20. The method of claim 14 wherein, prior to inserting the venous access device, the method further comprises the step of removing the vein visualization device from the skin of the patient after the desired portion of the venous structure is indicated.
